(19) United States
(12) Patent Application Publication
(10) Pub. No.: US 2024/0407950 A1
(43) Pub. Date: Dec. 12, 2024

(54) FAST RETINA TRACKING

(71) Applicant: PULSEMEDICA CORP., Edmonton, Alberta (CA)

(72) Inventors: Nir Katchinskiy, Edmonton, Alberta (CA); Tomoko Kurotobi, Edmonton, Alberta (CA)

(73) Assignee: PULSEMEDICA CORP., Edmonton, Alberta (CA)

(21) Appl. No.: 18/702,781

(22) PCT Filed: Oct. 21, 2022
(86) PCT No.: PCT/CA2022/051556

§ 371 Date: Apr. 18, 2024

(30) Foreign Application Priority Data
Oct. 22, 2021 (CA) 3135405

(51) Int. Cl. A61F 9/008; G06T 7/246

(52) U.S. Cl. CPC A61F 9/008; G06T 7/248; A61F 2009/00846; A61F 2009/00851; A61F 2009/00878; A61F 2009/00885; A61F 2009/00887; A61F 2009/00891; A61F 2009/00897; G06T 2207/10016; G06T 2207/10101; G06T 2207/30041

(57) ABSTRACT

Tracking of a patient's retina, or eye movements, may be useful when treating the patient's eye. The tracking may be done using an imaging system and may use two tracking processes. A first tracking process may determine eye movement based on a comparison between full frames of images. The second tracking process determines eye movement based on partial frames, or strips, of the images. The first tracking process must wait until the full frame is received, while the second tracking process may be performed quicker as it only requires a portion of the image to be received.

### RELATED APPLICATIONS

The current application claim priority to Canadian patent application 3,135,405 filed Oct. 22, 2021 entitled “FAST RETINA TRACKING,” the entire contents of which are incorporated herein by reference.

### TECHNICAL FIELD

The current disclosure relates to retina tracking. More specifically, in some embodiments, the present application is directed to systems and methods for tracking retina movement across multiple frames using full frame and sub-frame tracking.

### BACKGROUND

Imaging of an eye is important for identifying, and possibly treating, conditions of the eye. Various imaging techniques may be used for capturing images of the interior compartments of the eye. For example, scanning laser ophthalmoscopy (SLO) imaging may provide a 2-dimensional image of a portion of the eye, such as the retina or the cornea. Optical coherence tomography (OCT) imaging may provide 3-dimensional and/or cross-section images of a portion of the retina or cornea. Other imaging techniques may be used for capturing an image of at least a portion of the fundus of the eye.

Imaging of the eye may be used for identifying eye conditions requiring treatment. Treatment of eye conditions may be performed using lasers, with the specific targeting location of the laser beam or pulse determined from the captured images.

Although movement of a patient's eye may be minimized during treatment, even minor movements of the patient's eye may result in the laser no longer being targeted at the desired treatment location. Retina tracking techniques exist that can be used to track the patient's eye movements, however the tracking techniques may be relatively slow, or may accumulate errors.

An additional, alternative, and or improved retina tracking method is desirable.

### SUMMARY

In accordance with the present disclosure, there is provided a method for tracking movement of a patient's eye using a scanning-based imager, the method comprising: receiving a current image strip of a current image frame, the current image strip captured from the scanning-based imager; determining if the current image frame is complete; when it is determined that the current image frame is not complete: processing the current image strip to track movement between the current image strip and a corresponding image strip of a previously processed image frame providing a relative frame transformation of the current image frame for transforming locations in the current image frame to corresponding locations in the previously processed image frame; setting a current transformation based on a combination of the relative frame transformation of the current image frame and an absolute frame transformation of the previously processed image frame transforming locations in the previously processed image frame to corresponding locations in an initial image frame; and when it is determined that the current image frame is complete: processing the current image frame to track movement between the current image frame and an initial image frame, the tracked movement providing an absolute frame transformation of the current image frame for transforming locations in the current image frame to corresponding locations in the initial image frame; and setting the current transformation based on the absolute frame transformation of the current image frame.

In a further embodiment of the method, processing the current image frame to track movement comprises using one or more of feature tracking, and phase correlation to determine one or more translations and rotations for transforming the locations in the current image frame to the corresponding locations in the initial image frame.

In a further embodiment of the method, processing the current image strip to track movement comprises using one or more of feature tracking, and phase correlation to determine one or more translations for transforming the locations in the current image frame to the corresponding locations in the previously processed image frame.

In a further embodiment of the method, the method further comprises: registering a treatment plan comprising one or more treatment locations of the patient's eye with the initial image frame; and applying the current transformation to a next treatment location of the treatment plan to provide an adjusted next treatment location; and treating the next treatment location according to the treatment plan.

In a further embodiment of the method, treating the next treatment location comprises: adjusting one or more targeting and focusing elements of a laser delivery system to target the adjusted next treatment location; and firing the laser delivery system.

In a further embodiment of the method, the current image strip has a predefined number of rows of pixels captured by the scanning-based imager.

In a further embodiment of the method, a number of rows of pixels in the current image strip is determined dynamically.

In a further embodiment of the method, the number of rows of pixels in the current image strip is dynamically determined by: receive a next row of pixels of the current image strip; processing the current image strip to provide a trial relative frame transformation; determining if the trial relative frame transformation is determined with a high degree of confidence; and using the trial relative frame transformation as the relative frame transformation when the trial relative frame transformation is determined with the high degree of confidence.

In accordance with the present disclosure, there is further provided a non-transitory computer readable medium having instructions stored thereon which when executed by a processor of a computing device configure the computing device to perform a method according to any one of the embodiments of the method described above.

In accordance with the present disclosure, there is further provided a computing device comprising: a processor for executing instructions; and a memory having instructions stored thereon which when executed by the processor configure the computing device to perform a method according to any one of the embodiments of the method described above.

### BRIEF DESCRIPTION OF THE DRAWINGS

Further features and advantages of the present disclosure will become apparent from the following detailed description, taken in combination with the appended drawings, in which:

FIG. 1 depicts an imaging and laser treatment system incorporating retina tracking;

FIG. 2 depicts illustrative image frames and strips in accordance with the present method;

FIG. 3 depicts a method of retina tracking and ocular treatment;

FIG. 4 depicts a further method of retina tracking and treatment;

FIG. 5 depicts an illustrative timeline of the tracking process; and

FIG. 6 depicts a block diagram illustrating an embodiment of a computer hardware system configured to run software for implementing one or more embodiments of the health testing and diagnostic systems, methods, and devices disclosed herein.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present technology.

A fast retina tracking method may be used with linear scanning imaging devices that capture an image frame as a plurality of scan lines of the imaging target such as SLO and/or OCT. The fast retina tracking includes full-frame tracking that can track movement between full image frames, and sub frame tracking that tracks movement across strips of a frame or streams of image data. The full-frame tracking can wait until a complete new frame is captured from the scanning image device and can determine the movement between the complete new frame and a reference frame, such as an initial captured frame of the patient's eye used to register a treatment plan to the patient's eye position. The full-frame tracking process can provide an accurate determination of the patient's eye movement, including accounting for various translations and rotations of the eye.

However, full frame-tracking alone can be relatively slow, not only because a speed of the full-frame tracking depends on when a full frame is available but also because feature detection, matching and transformation determination can be computationally expensive when performed on an entire frame.

The strip tracking, or sub-frame tracking, process can track movement of an eye, retina, vitreous floaters, pupil, lens, sclera, cornea, and/or any other location or structure of the eye by identifying movement of one or more features across a strip of a frame currently being captured and a corresponding strip of a previously captured frame, or frames. The strip tracking can process image strips by identifying one or more features in the strip of the frame currently being captured and match the one or more features to corresponding one or more features in a corresponding strip of the previously captured frame to determine movement of the one or more features across strips of the previously captured frame and the frame currently being captured. The strip tracking process can determine translations of matching one or more features across strips, or may use other techniques to determine movement of matching one or more features between corresponding strips of different frames including for example correlation techniques such as phase correlation. Computing translations using strip tracking or sub-frame tracking can take less time than computing translations using full frame tracking because strip tracking or sub-frame tracking can use less degrees of freedom to compute translations than full frame tracking. However, by using less degrees of freedom and images strips to compute translations, strip tracking or sub-frame tracking can include more errors or be less accurate than full frame tracking.

By using full frame tracking in combination with strip tracking or sub-frame tracking a speed of eye movement tracking using linear imaging scanning devices can be significantly increased. For example, the linear imaging scanning device may capture a full frame every 32 ms and may capture a strip every 2 ms. Therefore, eye movement can be tracked about every 2 ms using strip tracking or sub-frame tracking. To reduce or eliminate the errors of strip tracking or sub-frame tracking, full frame tracking can be used to compare a full frame to an initial frame eliminating or reducing the error of the strip tracking or sub-frame tracking every 32 ms. It is to be appreciated that although strip tracking or sub-frame tracking and full frame tracking are described with reference to specific speeds, the specific speed are merely examples and are not intended to limit the scope of the disclosure.

Without using strip tracking or sub-frame tacking, tracking retina movement is limited to a speed at which a linear imaging scanning device can capture a full frame, and without using full frame tracking, an error from strip tracking or sub-frame tracking can cause a later treatment system to be more inaccurate over time.

Fast retina tracking can be performed by a computing system connected to or included in an imaging and laser delivery device. The fast retina tracking can be performed by a graphics processing unit (GPU), or custom hardware configured specifically to perform fast retina tracking to increase a speed or decrease a time needed to perform fast retina tracking.

The fast retina tracking process can be used in various applications, including for example for tracking eye movements to determine a location for targeting a laser a laser eye treatment system. The fast retina tracking can additionally or alternatively stabilize or align a stream of captured image frames by determining translations of stationary features within the eye and automatically aligning the stationary features across the stream of captured images. Stabilizing or aligning the stream of captured images can increase detection of or movement of moving structures across the stream of captured images such as floaters. The fast retina tracking process can be used to calculate a translation or displacement of one or more features within the eye to correct for motion of the eye or the one or more features within the eye across a stream of captured images. The calculated translation or displacement can be used as an automatic safety. The automatic safety can automatically turn off one or more components of the laser eye treatment system or stop one or more functions of the one or more components of the laser eye treatment system. For example, if the calculated translation or displacement is above a threshold, the laser treatment system can prevent or stop a firing of a laser. Although described with particular reference to laser eye treatment systems, the fast retina tracking process may be used in any application in which images of an eye are captured using a scanning imaging device.

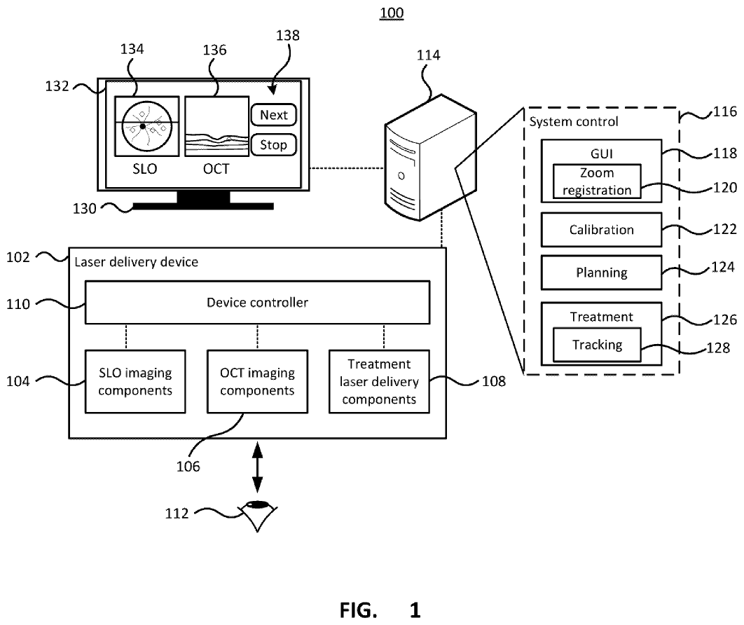

FIG. 1 depicts an imaging and laser treatment system 100 incorporating retina tracking. The system 100 can include an imaging and laser delivery device 102. The imaging and laser delivery device 102 can include SLO imaging components 104, OCT imaging components 106 and/or treatment laser delivery components 108. In some embodiments, the SLO imaging components 104, the OCT imaging components 106 and treatment laser delivery components 108 can include focusing components, light generating components, image sensors and/or any other components. The SLO imaging components 104, the OCT imaging components 106 and/or the treatment laser delivery components 108 can be controlled by a device controller 110. A light generated by the SLO imaging components 104, a light generated by the OCT imaging components 106 and/or a light or laser generated by the treatment laser delivery components 108 can be delivered to an eye 112, or possibly other target, being imaged and/or treated. The light generated by the SLO imaging components 104 and/or the OCT imaging components 106 can reflected back from a portion of the eye 112, such as a retina, to detectors of the corresponding imaging components.

The device controller 110 can be in wired or wireless communication with a computing device 114 such that the device controller is an interface between the imaging and laser delivery device 102 and the computing device 114. In some embodiments, the computing device 114 operates the imaging and laser delivery device 102 via system control functionalities 116. While the computing device 114 is depicted as a separate computing device 114, in some embodiments, the computing device 114 can be part of or incorporated into the imaging and laser delivery device 102. In some embodiments, the SLO imaging components 104 and/or the OCT imaging components 106 can transmit data to the device controller 110. The data can include location data, one or more coordinates, image data, depth data, an orientation of one or more mirrors of the SLO imaging components 104 and/or the OCT imaging components 106, or any other data.

The computing device 114 can include one or more processing units (not depicted) for executing instructions, and one or more memory units (not depicted) for storing data and instructions. The one or more processing units can execute the instruction to operate the imaging and laser delivery device 102 via the system control functionalities 116. In some embodiments, the one or more processing units can include a graphics processing unit (GPU), a central processing unit (CPU), a field-programmable gate array (FPGA), an application-specific integrated circuit (ASIC), a microcontroller (MCU), and/or any other hardware processing unit. The system control functionalities 116 can include graphical user interface (GUI) functionality 118 that provides a GUI for operating the imaging and laser delivery device.

As depicted, the GUI functionality 118 can include zoom registration functionality 120. A user can use the zoom registration functionality 120 to zoom in the SLO imaging components 104 and/or the OCT imaging components 106 and maintain registration of points on a zoomed in image with corresponding points of the other imaging components or laser delivery device 108. In some embodiments, the SLO imaging components 104, the OCT imaging components 106 and/or the laser delivery device 108 can use one or more coordinate systems. The zoom registration functionality 120 can automatically perform transformations between coordinate systems in order to provide a registration across the SLO imaging components 104, the OCT imaging components 106 and the laser delivery device 108 For example, a first transformation can map zoomed-in coordinates of the SLO imaging components 104 to zoomed out coordinates of the SLO imaging device 104, while a second transformation can map the zoomed out coordinates of the SLO imaging components 104 to coordinates of the OCT imaging components 104. The GUI functionality 118 can display a zoomed-in view of an image captured by the SLO imaging components 104 to a user, and points in the zoomed-in view can be transformed to corresponding points in a displayed view of an image captured by the OCT imaging components 106 by applying the first transformation and the second transformation to coordinates of the zoomed-in view of the image captured by the SLO imaging components 106. In some embodiments, the transformations can be predetermined transformations based on a zoom level of the SLO imaging components 104 and/or a zoom level of the OCT imaging components 106. In some embodiments, the zoom registration functionality 120 can dynamically determine the transformations in substantially real time based on the zoom level of the SLO imaging components 104 and/or the zoom level of the OCT imaging components 106. In some embodiments, the zoom registration functionality 120 can maintain registration of points on a zoomed in SLO image with corresponding points of the laser delivery device 108, and/or the zoom registration functionality 120 can maintain registration of points on a zoomed in SLO image with corresponding points of the laser delivery device 108.

In some embodiments, the system control functionalities 116 can include a calibration functionality 122. The calibration functionality 122 can align and correlate the SLO imaging components 104, OCT imaging components 106 and the treatment laser delivery components 108 so that locations in images captured by the SLO imaging components 104 and images captured by the OCT imaging components are aligned and the laser delivery device 108 can accurately target the locations. In some embodiments, the calibration functionality 122 can use image processing techniques to align and correlate the SLO imaging components 104, OCT imaging components 106 and the treatment laser delivery components 108, or the calibration functionality 122 can use various sensors and actuators to physically align two or more of the SLO imaging components 104, OCT imaging components 106 and the treatment laser delivery components 108.

In some embodiments, the system control functionalities 116 can include a planning functionality 124. The planning functionality 124 can develop a treatment plan for treating an ocular condition. The planning functionality 124 can use the GUI functionality 118 and/or image data received from the SLO imaging components 104 and the OCT imaging components 106 to determine a treatment plan. In some embodiments, the planning functionality 104 can display via the GUI functionality 118 the image data and the planning functionality 104 can include one or more user controls and/or user inputs to allow a user to select one or more treatment locations in the image data. In some embodiments, the planning functionality 104 can use artificial intelligence and/or machine learning to automatically detect one or more ocular conditions in the image data. A location of the one or more ocular conditions in the image data can be the one or more treatment location.

In some embodiments, the system control functionalities 116 can include a treatment functionality 126. The treatment functionality can control the SLO imaging components 104, the OCT imaging components 106 and/or the treatment laser delivery components 108 to aim the treatment laser delivery components 108 at the one or more treatment locations. The treatment functionality 126 can include a tracking functionality 128 that can track movement of a patient's eye in order to accurately aim the treatment laser delivery components 108 at the one or more treatment locations as the patient's eye moves.

In some embodiments, the GUI functionality 118 can display a generated GUI 132 on a display device 130. Although depicted as a separate display, in some embodiments, the display device 130 can be part of or incorporated into the imaging and laser delivery device 102. In some embodiments, one or more portion of the generated GUI can vary depending upon what information needs to be, or may be desirable to be, displayed to the user. FIG. 1 depicts a GUI 132 that could be displayed during treatment. For example, the GUI 132 can display an SLO image 134, and an OCT image 136. The SLO image 134 can include an indication of a location of a cross section of the OCT image 136. In some embodiments, the SLO image 134 and/or the OCT image 136 can include indications of one or more treatment locations that have not yet been treated as well as treatment locations that have been treated. In some embodiments, the GUI 130 can include treatment plan information that may be relevant to the user as well as one or more graphical elements 138 for starting/stopping the treatment plan or proceeding with the treatment plan such as proceeding to a next treatment location.

The laser delivery device 102 and the system 100 depicted in FIG. 1 broadly comprise optical hardware, control electronics and software. Components of the laser delivery device 102 and the system 100 are described in further detail below. The system 100 may be used for imaging eyes to identify areas for treatment and carrying out the treatment. The imaging and possible treatment can be for a wide range of different ocular conditions including, for example, symptomatic vitreous opacities or vitreous floaters (SVO), age-related macular degeneration (AMD), vitreomacular traction syndrome (VTS), diabetic retinopathy, cataracts, choroidal neovascularization, micro-aneurysm, glaucoma, epiretinal membrane (ERM), retinal tears and detachment, central or branch vein occlusion.

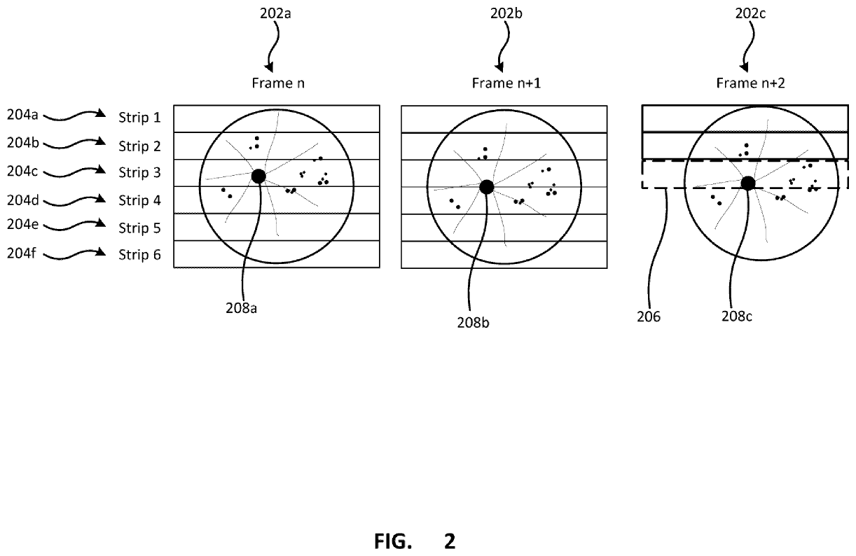

FIG. 2 depicts illustrative image frames and strips in accordance with the systems and methods disclosed herein. As described above, the fast retina tracking can be used to determine transformations or changes across a stream of captured images using full frame tracking, and real time or substantially real time sub-frame tracking via one or more strips of a full-frame as a system receives the one or more strips. As depicted, a first image frame 202a, a second image frame 202b, and a partial image frame 202c, can include a plurality of individual strips 204a-204f.

Each of the individual strips 204a-204f can include a number of row scans. The individual strips 204a-204f within the image frames 202a-202c are depicted as being a same size, or as having a same number of rows from the scanning device. It will be appreciated that the size of the individual strips 204a-204f can vary, both within a same frame as well as across different frames of the stream of captured images. In some embodiments, the size of each of the individual strips 204a-204f can be determined dynamically by a computer system based on various factors including for example, a processing speed and/or a processing load of the computer system of the computer system, which can determine how long processing a strip will take, a capture rate of imaging components for capturing a scan row, a region of the eye covered by the strip, features within the region of the eye covered by the strip, etc. A corresponding strip in a previous frame can be determined as a strip in the previous frame having a same, or similar, size and location in the previous frame as the size and location of a strip in the current frame. As shown by the partial image frame 202c, strips can be rows or portions of an image frame captured by imaging components. It is to be appreciate however, that the strips can a portion of the rows of the image frame captured by the imaging components, or the strips can be an entire row from of the image frame captured by the imaging components. In some embodiment, the strips can be a continuous data stream of image data captured by the imaging components.

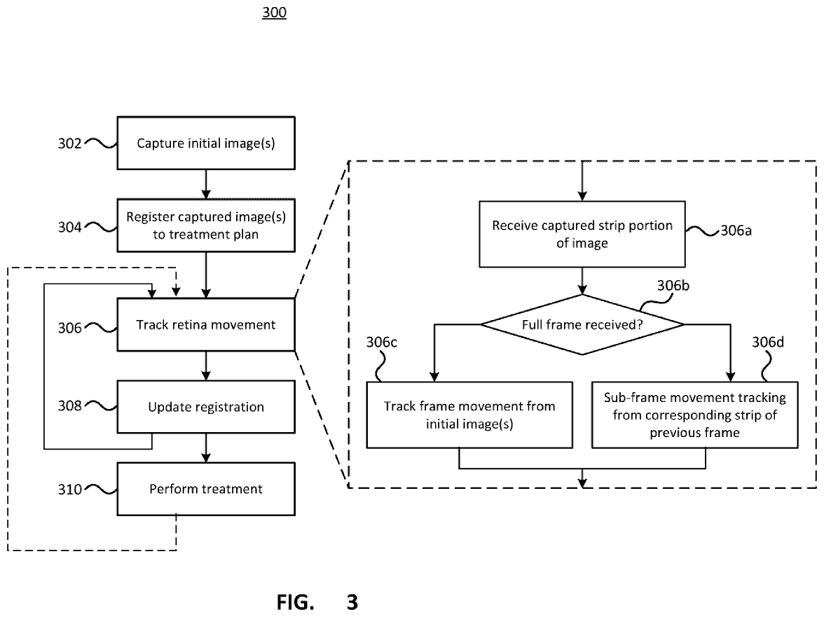

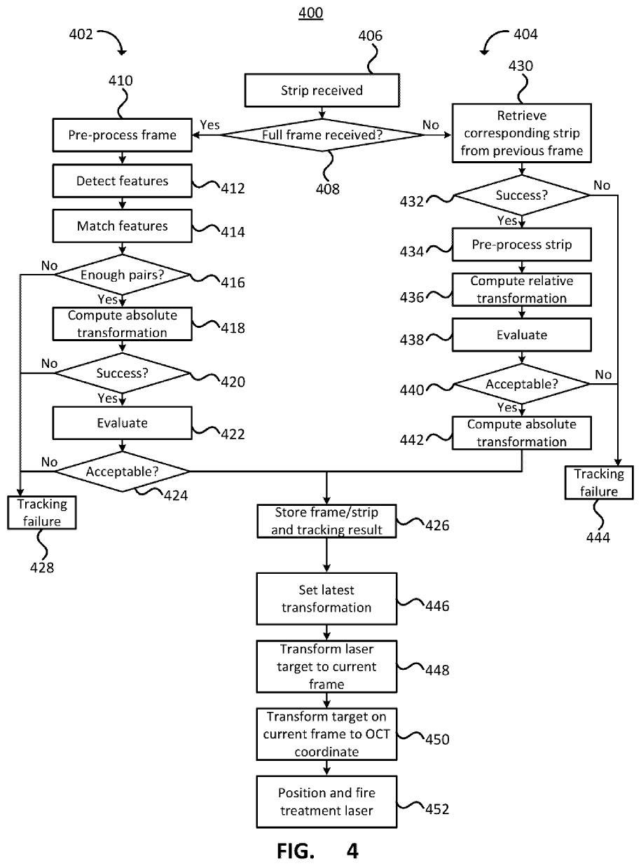

FIG. 2 depicts a current strip 206 as being captured. Although of the current image frame 202c is depicted as being captured from a top of the current image frame 202c to a bottom of the current image frame 202c, the imaging components can capture frames from bottom to top or side to side. In some embodiments, alternating image frames can be captured by the imaging components in opposite directions. For example, the imaging components can capture a first image frame from top to bottom, a second image frame from bottom to top, a third image frame from top to bottom, etc. In this way, the imaging components do not have to travel across the full image frame to start capturing a next frame. In some embodiments, the imaging components can capture each point of an image strip sequentially or the imaging components can capture an entire image strip at one time. The image frames can capture an image of the patient's eye and can be processed to identify movement of features 208a, 208b, 208c across the frames as described below with reference to FIGS. 3 and 4.

In some embodiments, the first image frame 202a can be captured by imaging components at a first time. In some embodiments, the imaging components can capture the entire first image frame 202a at the first time, or the imaging components can capture one or more of the plurality of individual strips 204a-204f at different times. For example, the imaging components can capture individual strip 204a of the first image frame 202a at the first time and individual strip 204b of the first image frame 202a at a second time after the imaging components capture the individual strip 204a of the first image frame 202a. In another example, the imaging components can capture individual strip 204a of the first image frame 202a and individual strip 204b of the first image frame 202a at the first time and the imaging components can capture one or more of uncaptured individual strips 204c-204f of the first image frame 202a at the second time. In some embodiments, after the imaging components capture the first image frame 202a, the imaging components can capture subsequent image frames 202b-202c.

In some embodiments, a plurality of imaging components can capture a plurality of individual strips 204a-204f at a same time. For example, the plurality of imaging components can be positioned or aimed such that the plurality of imaging components can each capture an individual strip or a portion of the individual strip of the plurality of individual strips 204a-204f of the first image frame 202a at a same time. In this way, a larger portion of the first image frame 202a or an entire first image frame 202a can be captured at the same time or substantially the same time.

In some embodiments, the imaging components can transmit image data of the plurality of individual strips 204a-204f and/or entire image frames 202a-202b to a computer system and/or a GPU of the computer system. The imaging components can transmit the image data to the computer system after the imaging components capture an entire individual strip 204a-204f, or the imaging components can transmit the image data of a portion of an individual strip 204a-204f to the computer system in real time or substantially real time as the imaging components capture the portion of the individual strip 204a-204f.

In some embodiments, the computer system can process received image data and perform fast retina tracking as described below with reference to FIGS. 3 and 4.

FIG. 3 depicts a method of retina tracking and ocular treatment. In some embodiments, method 300 can be performed by an imaging and laser treatment system as described herein with reference to FIG. 1. In some embodiments, method 300 can be performed by the imaging and laser treatment system after the imaging and laser treatment system and/or a user determine a treatment plan for the patient. The treatment plan can include one or more previously identified treatment locations of an ocular condition in previously captured images of a patient's eye. The method 300 begins with step 302 wherein the imaging and laser treatment system can capture initial image(s) of the patient's eye. At step 304, the imaging and laser treatment system can be registered to an orientation of the patient's eye based on the initial image(s) by identifying the treatment locations the initial images(s) so the imaging and laser treatment system can target treatment laser delivery components or a treatment laser at the predetermined locations for treatment of the ocular condition. Once the imaging and laser treatment system is registered to the orientation of the patient's eye, imaging and laser treatment system can track retina movement of the patient's eye at step 306 and at step 308 the retina movement can be used to determine an updated treatment location and update the registration of the imaging and laser treatment system. The tracking and updating at steps 306 and 308 can be performed continuously so that when treatment is performed, the imaging and laser treatment system is targeted at a current location of the ocular condition based on the retina movement tracked by the imaging and laser treatment system. The current location of the ocular condition can be the updated treatment location determined at step 308. At step 310, after updating the registration of the imaging and laser treatment system at step 308, the imaging and laser treatment system can treat the ocular condition at the updated treatment location, for example by firing a laser at the updated treatment location.

As depicted in FIG. 3, the imaging and laser treatment system can use fast retina tracking to track retina movement of the patient's eye at step 306. The fast retina tracking can include full frame tracking and/or strip, or sub-frame, tracking. The imaging and laser treatment system can include scanning imaging devices (e.g., SLO imaging devices, OCT imaging device, etc.) that generate or capture a full image frame from a plurality of individual strips or scans as described above with reference to FIG. 2. In some embodiments, each strip or scan can be a row of the full frame or a portion of a row of the full frame. The imaging and laser treatment system can use fast retina tracking to process strips, with each image strip including a number of rows captured by the scanning imaging device. That is, a full image frame can be formed by a plurality of strips which in turn can be formed by a plurality of row scans. A number of strips in the full frame and a size of the strips can be predefined or can be adjusted dynamically by the imaging and laser treatment system based on various factors including for example, a processing speed and/or a processing load of a computer system of the imaging and laser treatment system or a GPU of the computer system, a capture rate of the scanning imaging devices for capturing a scan row, a region of the eye covered by the strip, features within the region of the eye covered by the strip, etc..

In some embodiments, the imaging and laser treatment system can use steps 306a-306d to perform fast retina tracking. At step 306a, the computer system can receive a strip an image frame. As described above with reference to FIG. 2, the computer system can receive the strip of the image frame after the scanning imaging device captures the entire strip or the computer system can receive a portion of the strip in real time or substantially real time as the scanning imaging device captures the portion of the strip.

At step 306b, the computer system can automatically determine if the computer system received a full image frame or a strip of the image frame at step 306a. The computer system can receive location data from the scanning imaging device to determine if the computer system received a full image frame or a strip of the image frame. In some embodiments, the location data can include a position of a mirror of the scanning imaging device. The mirror of the scanning imaging device can direct a light or laser of the scanning imaging device to a location of the patient's eye. The computer system can determine when the location of the patient's corresponding to a location corresponding to the full image frame. In some embodiments, the location corresponding to the full image frame can be any corner or edge of the image frame. In some embodiments, the location corresponding to the full image frame can be any predetermined location in an image.

If the computer system received a full image frame at step 306a, the imaging and laser treatment system can use full frame tracking at step 306c to track retina movement of the patient's eye, as described below with reference to FIG. 4. If the computer system received a strip of the image frame at step 306a, the imaging and laser treatment system can use sub-frame tracking at step 306d to track retina movement of the patient's eye, as described below with reference to FIG. 4. At step 306c, the imaging and laser treatment system can track retina movement of the patient's eye by comparing the full frame to the initial image(s) and/or one or more image frames previously captured by the scanning imaging device after the initial images(s) were captured at step 302.

At step 306d, the imaging and laser treatment system can track retina movement of the patient's eye by comparing the strip of the image frame to the initial images, a corresponding strip in the initial images(s), one or more image frames previously captured by the scanning imaging device, and/or corresponding strips of the one or more image frames previously captured by the scanning imaging device. As described further below with reference to FIG. 4, at step 306d, the corresponding strips of one or more image frames previously captured by the scanning imaging device can be full image frames or portions of the one or more image frames that have already been captured by the scanning imaging device when the computer system receives the strip of the image frame. The retina movement of the patient's eye, whether determined from the full frame tracking or the strip tracking, may be used to update the registration of the imaging and laser treatment system at step 308.

FIG. 4 depicts a further method 400 of retina tracking and treatment. The method 400 can include a full-frame tracking process 402, and/or a sub-frame tracking process 404. In some embodiments, the sub-frame tracking process 404 can be a strip tacking process.

At step 406, a computer system can receive an image strip or image data of the image strip. At step 408, the computer system can automatically determine if a full frame has been received by the computer system. As described above with reference to FIG. 3, the computer system can receive location data from the scanning imaging device to determine if the computer system received a full image frame or a strip of the image frame. In some embodiments, the location data can include a position of a mirror of the scanning imaging device. The mirror of the scanning imaging device can direct a light or laser of the scanning imaging device to a location of the patient's eye. The computer system can determine when the location of the patient's corresponding to a location corresponding to the full image frame. In some embodiments, the location corresponding to the full image frame can be any corner or edge of the image frame. In some embodiments, the location corresponding to the full image frame can be any predetermined location in an image. If the computer system determines that a full frame has been received at step 408, the computer system can use the full-frame tracking process 402.

In some embodiments, the first step of the full-frame tracking process 402 can be step 410. At step 410 the computer system can pre-processes the full-frame or a portion of the full-frame. The computer system can applying one or more adjustments or transformations to the full-frame or the portion of the full-frame such as for example sharpening, adjusting white balance, colors, contrast or other image characteristics, removing lens distortions, etc.

After the computer system pre-processes the full frame at step 410, the computer system can detect one or more features of the patient's eye. The computer system can analyze the full frame to determine locations of the one or more features of the patient's eye in the full frame at step 412. The computer system can determine the locations of one or more veins, or other features of a retina of the patient's eye. The computer system can use various feature detection techniques or methods, including for example one or more of edge detection, corner detection, blob detection, and ridge detection.

After the computer system detects the one or more features of the patient's eye at step 412, the computer system can match the one or more features of the patient's eye to corresponding one or more features of the patient's eye of an initial frame at step 414. In some embodiments, the initial frame can be a first frame captured by a scanning imaging device, or the initial frame can be any previously received full image frame. After the computer system matches the one or more features of the patient's eye to corresponding features of the patient's eye in the initial frame at step 414, the computer system can determine if a threshold number of pairs of one or more features and corresponding one or more features have been matched at step 416. In some embodiments, the threshold number can be a predetermined number of pairs. The predetermined numbers of pair can be a number of pairs required to accurately transform the full image frame to line up with the initial frame. In some embodiments, the computer system can dynamically determine the threshold number of pairs depending on how much the computer system transformed a previously captured frame, a full frame capturing rate, a processing power of the computer system, etc. If the computer system determines enough pairs of features have been matched at step 416, the computer system can determine an absolute transformation at step 418. The absolute transformation can be translations, rotations, size changes, and/or warping applied to the full image frame such that one or more features of the full image frame line up with the corresponding one or more features of the initial image frame, or the one or more features of the full image frame are located at a same location as a location of the corresponding one or more features of the initial image frame

After the computer system determines the absolute transformation at step 418, the computer system can determine if the absolute transformation was computed successfully at step 420. If the computer system determines the absolute transformation was computed successfully at step 420, the computer system can evaluate the absolute transformation at step 422 to determine if the absolute transformation is above a transformation threshold at step 424. The transformation threshold can be a translation, a rotation, a size changes, and/or warping that when applied to a treatment laser would cause the treatment laser to fire as a line instead of a point. A transformation above the transformation threshold could cause unsafe firing of the treatment laser causing damage to the patient's eye. If at step 424, the computer system determines the absolute transformation is below the transformation threshold, the computer system can store the absolute transformation and/or a result of the full-frame tracking in a memory of the computer system at step 426.

If the computer system determines the threshold number of pairs of one or more features and corresponding one or more features have not been matched at step 416, the absolute transformation was not computed successfully at step 420, or the absolute transformation is above the transformation threshold at step 424, the computer system can indicate a tracking failure at step 428. In some embodiments, the tracking failure can prevent the treatment laser from firing.

If at step 408, the computer system determines the full frame has not been received when the computer system receives the image strip or image data of the image strip, the computer system can use the sub-frame tracking process 404. In some embodiments, the first step of the sub frame tracking process 404 can be step 430. At step 430, the computer system can retrieve a corresponding strip from a previously received full-image frame stored in a memory of the computer system. At step 432, the computer system can determine if the corresponding strip was retrieved successfully. If the computer system determines the corresponding strip was retrieved successfully, the computer system can pre-processes the image strip at step 434. The computer system can applying one or more adjustments or transformations to the image strip such as for example sharpening, adjusting white balance, colors, contrast or other image characteristics, removing lens distortions, etc.

After the computer system pre-processes the image strip at step 434, the computer system can compute a relative transformation at step 436. The computer system can compute the relative transformation by analyzing the image strip to determine locations of one or more features of the patient's eye in the image strip. The computer system can use various feature detection techniques or methods to determine locations of the one or more features of the patient's eye in the image strip, including for example one or more of edge detection, corner detection, blob detection, and ridge detection. After the computer system detects the one or more features of the patient's eye, the computer system can match the one or more features of the patient's eye to corresponding one or more features of the patient's eye of in the corresponding image strip. After the computer system matches the one or more features of the patient's eye to corresponding features of the patient's eye in the corresponding image strip, the computer system can determine if a threshold number of pairs of one or more features and corresponding one or more features have been matched. In some embodiments, the threshold number can be a predetermined number of pairs. The predetermined numbers of pair can be a number of pairs required to accurately transform the image strip to line up with the initial image strip. In some embodiments, the computer system can dynamically determine the threshold number of pairs depending on how much the computer system transformed a previously captured image strip, a image strip capturing rate, a processing power of the computer system, etc. If the computer system determines enough pairs of features have been matched, the computer system can determine a relative transformation. The relative transformation can be translations, rotations, size changes, and/or warping applied to the image strip frame such that one or more features of the image strip line up with the corresponding one or more features of the corresponding image strip, or the one or more features of the image strip are located at a same location as a location of the corresponding one or more features of the corresponding image strip. In some embodiments, the relative transformation can be a simplified transformation when compared to the absolute transformation. For example, in some embodiments, the relative transformation can include only translations. In this way, the computer system can calculate the relative transformation in less time than the computer system can calculate the absolute transformation. The relative transformation can be less accurate than the absolute transformation.

In some embodiments, the computer system can evaluate the elative transformation at step 438, to determine, at step 440, if an error of the relative transformation is within an acceptable error range and/or if the relative transformation is below the transformation threshold. The computer system can determine the error of the relative transformation by comparing the locations of the one or more features of the corresponding image strip to the location of the one or more features of the image strip after the relative transformation is applied to the image strip. It at step 440, the computer system determines the error of the relative transformation is within the acceptable error range and/or the relative transformation is below the transformation threshold, the computer system can determine a sub-tracking absolute transformation at step 442. The sub-tracking absolute transformation can be a combination of the relative transformation and a sub-tracking absolute transformation a previous image strip uses for the sub-tracking process 404, or the sub-tracking absolute transformation can be a combination of the relative transformation and an absolute transformation determined in a previous full frame tracking process 410. After the computer system determines the sub-tracking absolute transformation at step 442, the computer system can store the sub-tracking absolute transformation and/or a result of the sub frame tracking in a memory of the computer system at step 426.

If the computer system determines the corresponding strip was not retrieved successfully at step 432, or the error of the relative transformation is not within the acceptable error range and/or the relative transformation is above the transformation threshold, the computer system can indicate a tracking failure at step 428. In some embodiments, the tracking failure can prevent the treatment laser from firing.

After the computer system stores the absolute transformation or sub-tracking absolute transformation at step 426, the computer system can set the absolute transformation or sub-tracking transformation a latest transformation at step 446. In some embodiments, the computer system can apply the absolute transformation or the sub-tracking absolute transformation to laser treatment coordinates of a laser target of the current frame to transform a position of the laser target. After the computer system transforms applies the absolute transformation or the sub-tracking absolute transformation to the laser treatment coordinates, the computer system can apply the absolute transformation or the sub-tracking absolute transformation to coordinate of an OCT imaging device at step 450. Once the laser target is transformed, the treatment laser may be positioned and fired at step 452.

Although not depicted, in some embodiments, the method 400 can be a feedback loop and the computer system can continuously perform method 400 as the computer system receives image strips. Continuous tracking of eye movement of the feedback loop can ensure that the treatment laser is positioned or aimed at a correct treatment location of the patient's eye as the patient's eye moves. It is to be appreciated that although steps 426-452 are described with reference to the treatment laser, method 400 can be used to ensure that a scanning imaging, such as an OCT imaging device, is position or aimed at a correct location.

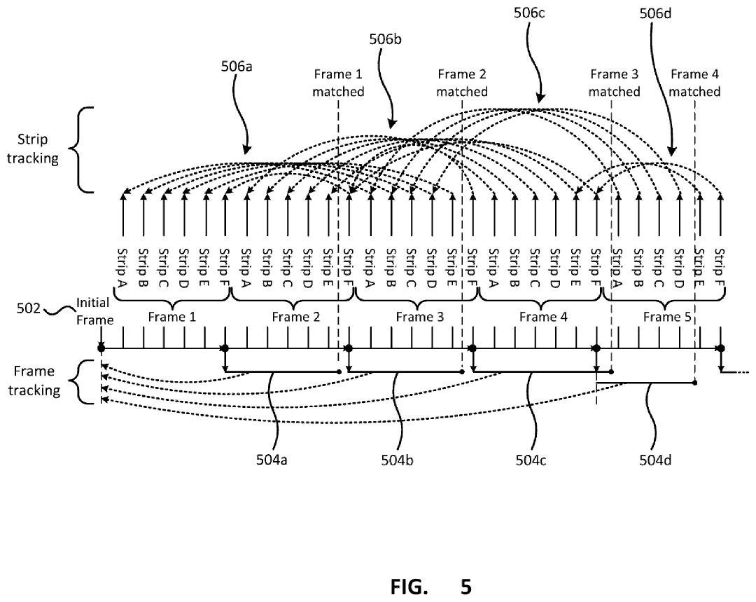

FIG. 5 depicts an illustrative timeline of the tracking process. As depicted, an initial frame is received 502. Although not depicted, the initial frame can be captured or imaged as a number of strips similar to additional frames. As depicted, each frame 1-5 can be captured or imaged as a series of strips A-F. A scanning imaging device can capture each strip as a series of points or the scanning imaging device can capture an entire strip at one time. When a computer system receives one of the series of strips A-F, the computer system can use the strip A-F and a corresponding strip A-F of a previous frame to perform sub-frame tracking, as depicted by arrows 506a-506d and described above with reference to FIG. 4. The corresponding strop A-F of the previous frame can be a corresponding strip of a most recently matched full-image frame.

Although the strips A-F of the Frame1 and Frame2 received before the Frame1 is matched are not shown as being matched to the initial frame 502 the strips A-F may be matched to corresponding strips of the initial frame.

As depicted the computer system can take more time to perform full frame tracking 504a-504d, than the computer system takes to receive each a plurality of strip A-F and perform sub-frame tacking 506a-506d using the plurality of strips A-F. As depicted, after frame 1 is matched using full frame tracking, Strip F of Frame 2 is compared to Strip F of Frame 1 using sub frame tracking, and Strip A and Strip E of Frame 3 are compared to corresponding Strips A-E of Frame 1 using sub frame tracking as depicted by arrows 506a. Similarly, each of the strips may be compared to corresponding strips of the previously matched frames as depicted by arrows 506b, 506c, and 506d. The computer system can perform sub-tracking on a series of strips A-F while the computer system simultaneously performs full frame tracking using a most recently captured full frame.

The process described above provides fast retina tracking with the possible eye movement being updated as each strip is received. As described above with reference to FIG. 4, the sub tracking process can be significantly faster than full frame tracking, however, the sub tracking can include an error that can accumulate with each sub tracking absolute transformation applied. Once a full frame is received and the computer system can perform full frame tracking, the absolute transformation calculate during the full frame tracking process can be applied reducing or eliminating accumulation of the errors since movement in the full frame tracking process is determined using full frames instead of image strips.

It is to be appreciated that although a computer system is described above with reference to FIGS. 2-5, any portion of a computer system can perform any of the methods described herein. In some embodiments, a GPU can perform the methods described herein to increase a speed at which fast retina tracking can be performed. A GPU can use parallel processing to perform fast retina tracking. In some embodiments, a custom processor can be used to perform any of the methods described herein. The custom processor can include one or more GPUs, CPUs, FPGAs, ASICs, and/or any other hardware. In some embodiments, multiple GPUs and/or multiple custom processors can perform the methods described herein at a same time on different full frame images or image strips further increases a processing speed.

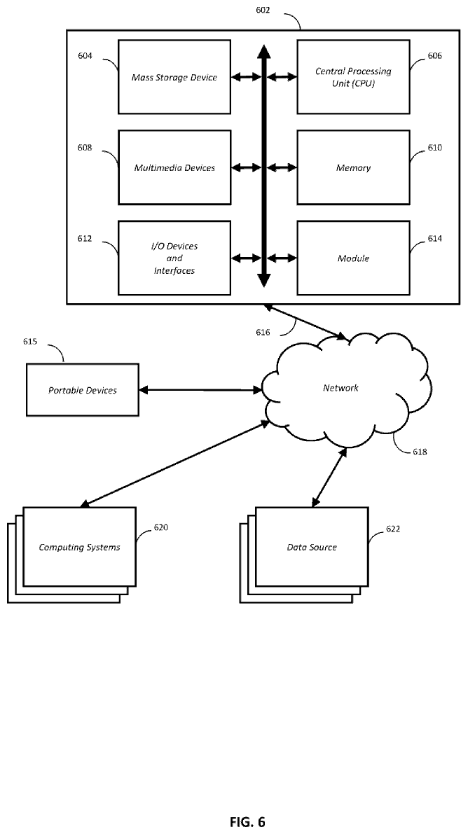

FIG. 6 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 6. The example computer system 602 is in communication with one or more computing systems 20 and/or one or more data sources 622 via one or more networks 618. While FIG. 6 illustrates an embodiment of a computing system 602, it is recognized that the functionality provided for in the components and modules of computer system 602 may be combined into fewer components and modules, or further separated into additional components and modules.

The computer system 602 can comprise a module 614 that carries out the functions, methods, acts, and/or processes described herein. The module 614 is executed on the computer system 602 by a central processing unit 606 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 602 includes one or more processing units (CPU) 606, which may comprise a microprocessor. The computer system 602 further includes a physical memory 610, such as random-access memory (RAM) for temporary storage of information, a read only memory (ROM) for permanent storage of information, and a mass storage device 604, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 602 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 602 includes one or more input/output (I/O) devices and interfaces 612, such as a keyboard, mouse, touch pad, and printer. The I/O devices and interfaces 612 can include one or more display devices, such as a monitor, that allows the visual presentation of data to a user. More particularly, a display device provides for the presentation of GUIs as application software data, and multi-media presentations, for example. The I/O devices and interfaces 612 can also provide a communications interface to various external devices. The computer system 602 may comprise one or more multi-media devices 608, such as speakers, video cards, graphics accelerators, and microphones, for example.

The computer system 602 may run on a variety of computing devices, such as a server, a Windows server, a Structure Query Language server, a Unix Server, a personal computer, a laptop computer, and so forth. In other embodiments, the computer system 602 may run on a cluster computer system, a mainframe computer system and/or other computing system suitable for controlling and/or communicating with large databases, performing high volume transaction processing, and generating reports from large databases. The computing system 602 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 602 illustrated in FIG. 6 is coupled to a network 618, such as a LAN, WAN, or the Internet via a communication link 616 (wired, wireless, or a combination thereof). Network 618 communicates with various computing devices and/or other electronic devices. Network 618 is communicating with one or more computing systems 620 and one or more data sources 622. The module 614 may access or may be accessed by computing systems 620 and/or data sources 622 through a web-enabled user access point. Connections may be a direct physical connection, a virtual connection, and other connection type. The web-enabled user access point may comprise a browser module that uses text, graphics, audio, video, and other media to present data and to allow interaction with data via the network 618.

Access to the module 614 of the computer system 602 by computing systems 620 and/or by data sources 622 may be through a web-enabled user access point such as the computing systems' 620 or data source's 622 personal computer, cellular phone, smartphone, laptop, tablet computer, e-reader device, audio player, or another device capable of connecting to the network 618. Such a device may have a browser module that is implemented as a module that uses text, graphics, audio, video, and other media to present data and to allow interaction with data via the network 618.

The output module may be implemented as a combination of an all-points addressable display such as a cathode ray tube (CRT), a liquid crystal display (LCD), a plasma display, or other types and/or combinations of displays. The output module may be implemented to communicate with input devices 612 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 602 may comprise a physical or logical connection established between a remote microprocessor and a mainframe host computer for the express purpose of uploading, downloading, or viewing interactive data and databases on-line in real time. The remote microprocessor may be operated by an entity operating the computer system 602, including the client server systems or the main server system, an/or may be operated by one or more of the data sources 622 and/or one or more of the computing systems 620. In some embodiments, terminal emulation software may be used on the microprocessor for participating in the micro-mainframe link.

In some embodiments, computing systems 620 who are internal to an entity operating the computer system 602 may access the module 614 internally as an application or process run by the CPU 606.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 602 may include one or more internal and/or external data sources (for example, data sources 622). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Caché), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

The computer system 602 may also access one or more databases 622. The databases 622 may be stored in a database or data repository. The computer system 602 may access the one or more databases 622 through a network 618 or may directly access the database or data repository through I/O devices and interfaces 612. The data repository storing the one or more databases 622 may reside within the computer system 602.

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

1-24. (canceled)

25. An imaging and treatment system for imaging and treating an eye condition comprising:
a scanning based imager arranged to image at least a portion of a patient's eye;
a treatment laser arranged to treat a patient's eye with a laser pulse;
a controller configured to:
capture image frames, using the scanning based imager, as a plurality of subsequently captured image strips;
perform image strip tracking to determine a movement of at least one feature in a most recently captured image strip relative to at least one corresponding feature in a corresponding image strip of a previously captured image frame; and
prevent the treatment laser from treating the patient's eye when the determined movement of the at least one feature exceeds a safety threshold.

26. The imaging and treatment system of claim 25, wherein the controller is further configured to:
adjust a treatment location of the treatment laser within the patient's eye based on the movement of the at least one feature when the movement of the at least one feature does not exceed the safety threshold.

27. The imaging and treatment system of claim 26, wherein the treatment location is specified in a treatment plan that is registered to a captured image frame of the patient's eye.

28. The imaging and treatment system of claim 25, wherein the controller is further configured to track motion of at least one structures based on the determined movement of the at least one feature, the at least one structures comprising at least one of:
an eye;
a retina;
a floater;
a pupil;
a lens;
a scelra; and
a cornea.

29. The imaging and treatment system of claim 25, wherein the at least one feature comprises at least one stationary feature within the patient's eye.

30. The imaging and treatment system of claim 29, wherein the controller is further configured to stabilize a plurality of captured image frames based on the at least one stationary feature.

31. The imaging and treatment system of claim 30, wherein the controller is further configured to track movement of a floater across the stabilized plurality of captured image frames.

32. The imaging and treatment system of claim 25, wherein determining the movement of the at least one feature comprises determining a translation of the at least one feature.

33. The imaging and treatment system of claim 25, wherein the controller is further configured to:
determine if a current image frame being captured is complete;
when it is determined that the current image frame is not complete:
perform the image strip tracking to determine a relative frame transformation of the current image frame for transforming locations in the current image frame to corresponding locations in the previously captured image frame; and
set a current transformation based on a combination of the relative frame transformation of the current image frame and an absolute frame transformation of the previously captured image frame transforming locations in the previously captured image frame to corresponding locations in an initial image frame; and
when it is determined that the current image frame is complete:
determine an absolute frame transformation of the current image frame for transforming locations in the current image frame to corresponding locations in the initial image frame; and
set the current transformation based on the absolute frame transformation of the current image frame.

34. The imaging and treatment system of claim 33, wherein the relative frame transformation comprises one or more translations and the absolute frame transformation comprises one or more translations and rotations.

35. The imaging and treatment system of claim 34, wherein the controller is further configured to:
register a treatment plan comprising one or more treatment locations of the patient's eye with the initial image frame;
apply the current transformation to a next treatment location of the treatment plan to provide an adjusted next treatment location; and
treat the next treatment location according to the treatment plan.

36. The imaging and treatment system of claim 25, wherein the scanning based imager comprises at least one of:
a scanning laser ophthalmoscopy (SLO) imager; and
an optical coherence tomography (OCT) imager.

37. The imaging and treatment system of claim 25, wherein the eye condition treated using the imaging and treatment system comprises one or more of:
symptomatic vitreous opacities (SVO);
floaters;
age-related macular degeneration (AMD);
vitreomacular traction syndrome (VTS);
diabetic retinopathy;
cataracts;
choroidal neovascularization;
micro-aneurysm;
glaucoma;
epiretinal membrane (ERM);
retinal tears and detachments; and
central or branch vein occlusions.

38. A method for tracking movement of a patient's eye using a scanning based imager of an imaging and treatment system during a laser eye treatment session, the method comprising:
capturing image frames, using the scanning based imager, as a plurality of subsequently captured image strips;
performing image strip tracking to determine a movement of at least one feature in a most recently captured image strip relative to at least one corresponding feature in a corresponding image strip of a previously captured image frame; and
preventing a treatment laser from treating the patient's eye when the determined movement of the at least one feature exceeds a safety threshold.

39. The method of claim 38, further comprising:
adjusting a treatment location of the treatment laser within the patient's eye based on the movement of the at least one feature when the movement of the at least one feature does not exceed the safety threshold.

40. The method of claim 39, wherein the treatment location is specified in a treatment plan that is registered to a captured image frame of the patient's eye.

41. The method of claim 38, further comprising tracking motion of at least one structures based on the determined movement of the at least one feature, the at least one structures comprising at least one of:
an eye;
a retina;
a floater;
a pupil;
a lens;
a scelra; and
a cornea.

42. The method of claim 38, wherein the at least one feature comprise at least one stationary feature within the patient's eye.

43. The method of claim 42, further comprising stabilizing a plurality of captured image frames based on the at least one stationary feature.

44. The method of claim 43, further comprising tracking movement of a floater across the stabilized plurality of captured image frames.

45. The method of claim 38, wherein determining the movement of the at least one feature comprises determining a translation of the at least one feature.

46. The method of claim 38, further comprising:
determining if a current image frame being captured is complete;
when it is determined that the current image frame is not complete:
performing the image strip tracking to determine a relative frame transformation of the current image frame for transforming locations in the current image frame to corresponding locations in the previously captured image frame; and
setting a current transformation based on a combination of the relative frame transformation of the current image frame and an absolute frame transformation of the previously captured image frame transforming locations in the previously captured image frame to corresponding locations in an initial image frame; and
when it is determined that the current image frame is complete:
determining an absolute frame transformation of the current image frame for transforming locations in the current image frame to corresponding locations in the initial image frame; and
setting the current transformation based on the absolute frame transformation of the current image frame.

47. The method of claim 46, wherein the relative frame transformation comprises one or more translations and the absolute frame transformation comprises one or more translations and rotations.

48. The method of claim 47, further comprising:
registering a treatment plan comprising one or more treatment locations of the patient's eye with the initial image frame;
applying the current transformation to a next treatment location of the treatment plan to provide an adjusted next treatment location; and
treating the next treatment location according to the treatment plan.

49. The method of claim 38, wherein an eye condition treated using the imaging and treatment system comprises one or more of:
symptomatic vitreous opacities (SVO);
floaters;
age-related macular degeneration (AMD);
vitreomacular traction syndrome (VTS);
diabetic retinopathy;
cataracts;
choroidal neovascularization;
micro-aneurysm;
glaucoma;
epiretinal membrane (ERM);
retinal tears and detachments; and
central or branch vein occlusions.

50. A non-transitory computer readable medium having instructions stored thereon which when executed by a processor configure a computing device to perform a method comprising:
capturing image frames, using a scanning based imager, as a plurality of subsequently captured image strips;
performing image strip tracking to determine a movement of at least one feature in a most recently captured image strip relative to at least one corresponding feature in a corresponding image strip of a previously captured image frame; and
preventing a treatment laser from treating a patient's eye when the determined movement of the at least one feature exceeds a safety threshold.
